(19) United States
(12) Patent Application Publication
(10) Pub. No.: US 2022/0039994 A1
(43) Pub. Date: Feb. 10, 2022

(54) COLLECTOR MEANS FOR USE WITH A URINE CATHETER FOR A MAN

(71) Applicant: RUDOLF HEINTEL GESELLSCHAFT M.B.H., Wien (AT)

(72) Inventors: Wilhelm Bauer, Klosterneuburg (AT); Markus Pöltenstein, Wien (AT)

(21) Appl. No.: 17/420,439

(22) PCT Filed: Jan. 10, 2020
(86) PCT No.: PCT/EP2020/050481

§ 371 Date: Jul. 2, 2021

(30) Foreign Application Priority Data
Jan. 11, 2019 (EP) 19151373.8

(51) Int. Cl. A61F 5/453; A61F 5/44; A61M 25/00; A61M 25/02

(52) U.S. Cl. CPC A61F 5/453; A61F 5/4401; A61M 2025/0253; A61M 25/02; A61M 25/0017

(57) ABSTRACT

The invention relates to a pad (1) for use with a urine catheter (2) for a man, comprising an element (3) made of absorbent material (4) and a liquid-tight layer (5) on one side, and at least one adhesive element (6) for fastening the element (3) in a use position and for fastening the element (3) in its use position on the urine catheter (2). According to the invention, the element (3) made of absorbent material (4) is in the form of a circle sector and, for its use position, can be rolled up to form a funnel and can be fixed in position using the at least one adhesive element (6), and at least one additional adhesive element (7) is arranged on a radius (r) of the circle sector for fastening to the urine catheter (2), which additional adhesive element (7) has an adhesive-free end (8).

### TECHNICAL FIELD OF THE INVENTION

The invention relates to a pad for use with a urine catheter for a man, comprising an element made of absorbent material and a liquid-tight layer on one side, and at least one adhesive element for fastening the element in a use position and for fastening the element in its use position on the urine catheter.

### BACKGROUND OF THE INVENTION

Urine catheters or transurethral bladder catheters are inserted into the bladder via the urethra and are held there by an inflatable balloon. Depending on the intended use, a distinction is made between permanent and disposable transurethral catheters. The length of the catheters is usually given in centimetres, and the thickness in Charrière (1 Ch=⅓ mm).

Especially when urine catheters are used in male patients, urine leaks from the outside of the catheter, resulting in soiling. In addition, infections are a further complication.

To avoid or minimise this problem, collection bags or the like are connected at the outer end of the urine catheter. Documents ES 2 342 647 A1 or DE 102 46 570 A1 describe such devices or catheter inserts.

For example, DE 10 2012 020 721 A1 describes a catheter in which a flat pad made of a liquid-binding material is arranged on the outer part of the catheter tube to prevent infections. The pad receives urine that leaks from the outside of the catheter. The pad can be fixed with a clamp that can be attached to the tube or with an adhesive tape.

Document U.S. Pat. No. 5,980,507 A describes an attachment for a urine catheter for a man for collecting urine, having an absorbent body and a fastening unit which removably arranges the absorbent body around the catheter tube by means of adhesive strips. In addition, it is possible to fix the pad to the catheter tube using adhesive strips.

Previous pad for use with a urine catheter for a man are usually elaborate in their manufacture and/or use. There is therefore a need for a product that is relatively inexpensive and easy to manufacture.

### SUMMARY OF THE INVENTION

The object of the present invention is therefore to create a pad of the kind mentioned above for use in a urine catheter for a man which is as simple and inexpensive to manufacture as possible. The pad should be as quick and easy to use as possible and should also be removable in order to guarantee a broad application and in order to be able to comply with the hygiene conditions. Disadvantages of known products should be avoided or at least reduced.

The object according to the invention is solved in that the element made of absorbent material is in the form of a circle sector and, for its use position, can be rolled up to form a funnel and can be fixed in position using the at least one adhesive element, and at least one additional adhesive element is arranged on a radius of the circle sector for fastening to the urine catheter, which additional adhesive element has an adhesive-free end. This design of the pad guarantees simple and inexpensive manufacture, since the main component of the pad, i.e. the element in the form of a circle sector and made of absorbent material, can be produced simply from a flat material web. The application of the adhesive elements to the absorbent material can also be carried out particularly quickly and easily, even in automated fashion. It is also possible to use the pad by forming the element in the form of a circle sector into a funnel around the catheter and by fixing the funnel using the adhesive tape. The funnel can be fixed at the desired position on the catheter tube using the at least one further adhesive element. Due to the adhesive-free end of the at least one additional adhesive element, this element can be removed very quickly and easily, and the pad can be disposed of or replaced. The pad according to the invention is characterised by the possibility of particularly low-cost manufacture and simple use. Of course, deviations of the element made of absorbent material from the form of a circle sector are possible. For example, the absorbent material can also be used in the form of part of a polygon.

Advantageously, the element in the form of a circle sector has a central angle of the circle sector of 90° to 270°, preferably 180°. Depending on the size or opening angle of the funnel formed by the element in the form of a circle sector the central angle can be selected accordingly. With the preferred central angle of 180°, the waste when manufacturing the element from the absorbent material can be kept particularly low.

Ideally, the radius of the element in the form of a circle sector is between 5 cm and 10 cm. These dimensions have proven to be particularly suitable when using the pad with a urine catheter for a man.

The area of the element in the form of a circle sector is preferably between 20 cm² and 200 cm². In addition to the thickness of the absorbent material, the area of the element in the form of a circle sector determines the absorption capacity for the urine to be absorbed.

If a cut-out is provided in the region of the centre of the element in the form of a circle sector, the urine catheter can be ideally surrounded in the use position of the pad, and folds that might result in an escape of urine flowing on the outside of the catheter tube can be avoided.

The cut-out is preferably circular with a radius of preferably 3 mm to 11 mm, corresponding to the usual diameters of urine catheters for men in the range of 11 to 34 Charrière.

Instead of a cut-out, at least one incision can be provided in the region of the centre of the element in the form of a circle sector. By means of at least one such incision, an improved connection of the pad to the catheter can also be achieved. An incision is particularly easy to make.

This at least one incision preferably has a depth of 3 mm to 11 mm, corresponding to the above-mentioned usual catheter diameters.

The element in the form of a circle sector can be made of highly absorbent plastic fibres, in particular acrylic or polyester fibres. Such materials are known from wound dressings and allow the absorption of relatively large amounts of bodily liquids.

In addition, a gel-forming substance can be arranged in the element in the form of a circle sector in order to bind any absorbed urine.

A contact mesh may be arranged on the side of the element in the form of a circle sector opposite the liquid-tight layer. Such a contact mesh has pores or openings to receive the urine but prevents the skin from sticking to the absorbent material.

The adhesive element for fastening the element in the form of a circle sector in its use position can, for example, be formed by a rounding. This is shaped in such a way that there is no overlap or protrusion when the element in the form of a circle sector is rolled into a funnel. The adhesive element must be of such a size that the funnel can be fixed to the catheter as quickly and easily as possible.

The adhesive elements, that is to say the adhesive element for fastening the element in the form of a circle sector in the use position and the additional adhesive element for fastening to the urine catheter, can be formed in one piece. This facilitates the manufacture of the pad and thereby also reduces the manufacturing costs.

The thickness of the element in the form of a circle sector can be 1 to 9 mm, preferably 3 to 3.5 mm. The thickness depends on how many layers of absorbent material are used.

### BRIEF DESCRIPTION OF THE DRAWINGS

The present invention will be explained in more detail with reference to the accompanying drawings, in which:

FIG. 1 shows a plan view of an embodiment of a pad for use in a urine catheter for a man;

FIG. 2 shows a sectional view through the element in the form of a circle sector of the pad along line of section II-II in FIG. 1;

FIG. 3A shows a detail of the pad in the region of the centre M of the element in the form of a circle sector with a cut-out;

FIG. 3B shows a detail of the pad in the region of the centre M of the element in the form of a circle sector with an incision; and

FIG. 4 shows the pad according to FIG. 1 in its use position, fastened to the urine catheter for a man.

### DETAILED DESCRIPTION OF THE INVENTION

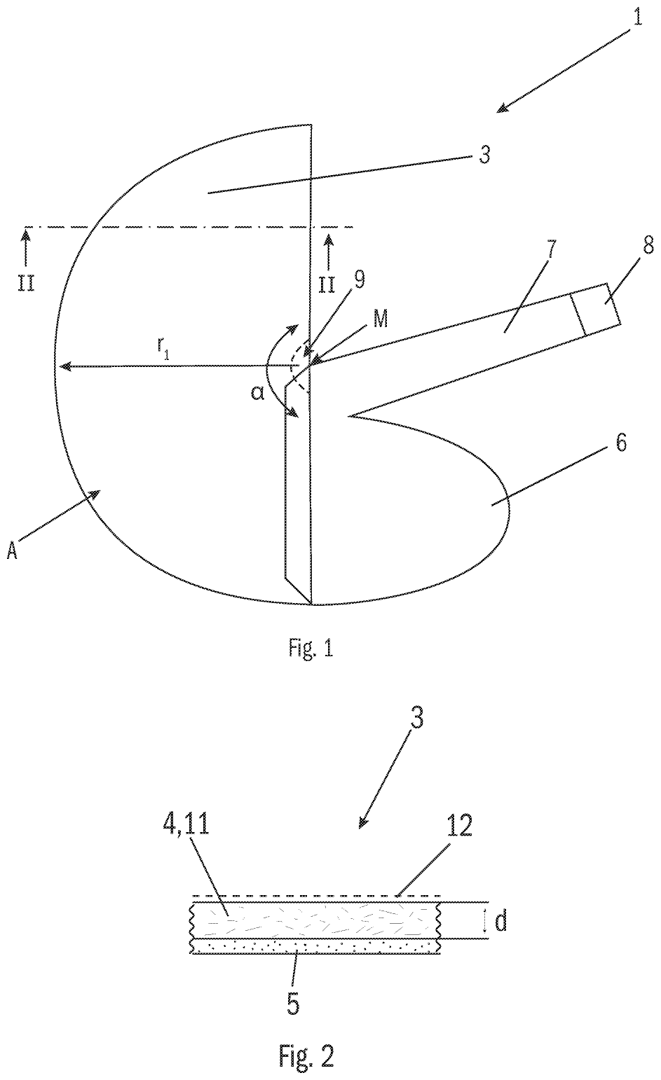

FIG. 1 shows a plan view of an embodiment of a pad 1 for use with a urine catheter 2 for a man. The pad 1 has an element in the form of a circle sector 3, which in the illustrated embodiment example has a central angle α of 180°. Other central angles α between 90° and 270° are also possible. Suitable sizes for the element in the form of a circle sector 3 include a circle radius r₁ between 5 cm and 10 cm and an area A between 20 cm² and 200 cm². The pad 1 or the element in the form of a circle sector 3 can be formed into a funnel and fastened in the use position by means of at least one adhesive element 6 (see FIG. 4) The pad is fastened to the urine catheter 2 by means of at least one further adhesive element 7, which can be formed in one piece with the adhesive element 6. To facilitate removal of the pad 1 from the urine catheter, the further adhesive element 7 has an adhesive-free end 8. The adhesive layers of the adhesive elements 6, 7 are usually protected by a peel-off film (not shown), which is peeled off before use.

FIG. 2 shows a sectional view through the element in the form of a circle sector 3 of the pad 1 along the line of section II-II in FIG. 1. Accordingly, the element in the form of a circle sector 3 of the pad 1 consists of absorbent material 4 and a liquid-tight layer 5 on one side. On the side opposite the liquid-tight layer 5, there may be arranged a contact mesh 12, which allows the absorption of liquid into the layer of absorbent material 4 but prevents adhesion or sticking to the absorbent material 4. The absorbent material 4 of the element in the form of a circle sector 3 may be formed of highly absorbent plastic fibres, especially acrylic or polyester fibres. Furthermore, a gel-forming substance 11 may be arranged in the absorbent material. The thickness d of the element in the form of a circle sector 3 is, for example, 1 mm to 9 mm, preferably 3 mm to 3.5 mm.

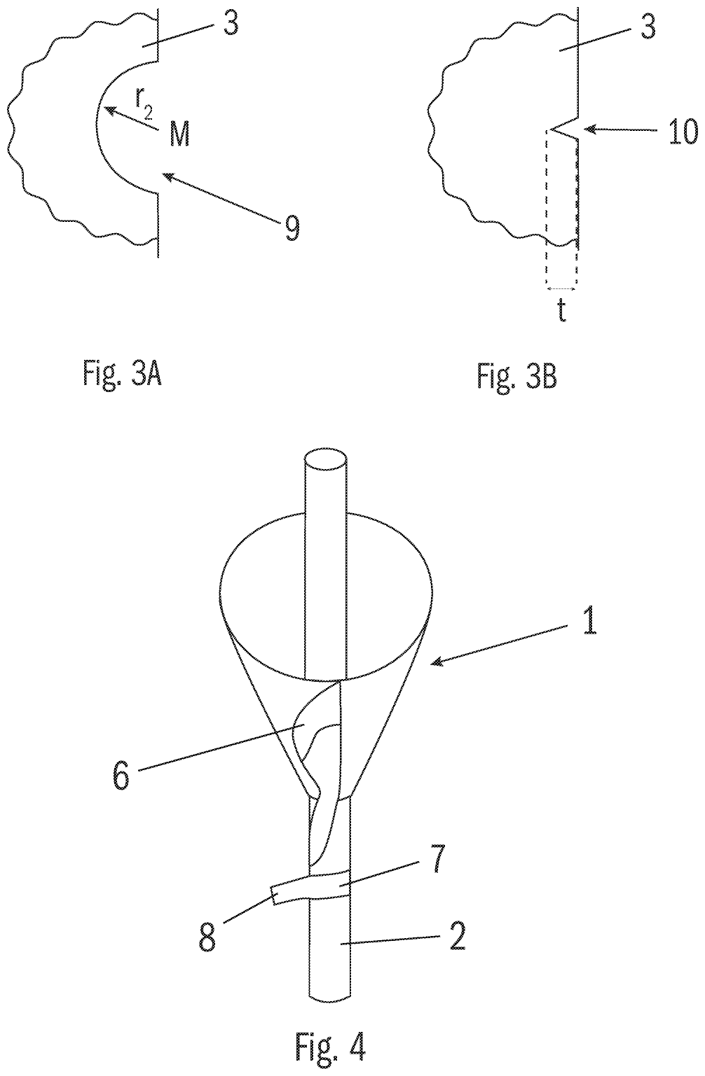

FIG. 3A shows a detail of the pad 1 in the region of the centre M of the element in the form of a circle sector 3 with a circular cut-out 9. The cut-out 9 is preferably circular, with a radius r₂ of preferably 1 mm to 11 mm, corresponding to the common diameter of the urine catheter 2 from 11 to 34 Charrière.

FIG. 3B shows a variant of an incision 10 in the region of the centre M of the element in the form of a circle sector 3. This incision 10 preferably has a depth t of 1 mm to 11 mm.

Lastly, FIG. 4 shows the pad 1 according to FIG. 1 in its use position fastened to the urine catheter 2 for a man. When using the pad 1, the element in the form of a circle sector 3 is arranged around the urine catheter 2 and the funnel shape is fixed using the adhesive element 6. Once the pad has been moved to the desired position on the urine catheter 2, it is fixed to the urine catheter using the at least one further adhesive element 7. Since the at least one further adhesive element 7 has an adhesive-free end 8, the pad can also be detached and removed from the urine catheter 2 particularly quickly and easily.

The described pad 1 for use with the urine catheter 2 for a man is particularly easy and inexpensive to produce and particularly easy to use. For this reason, the pad 1 helps to improve hygiene and prevent infections.

1. A pad (1) for use with a urine catheter (2) for a man, comprising an element (3) made of absorbent material (4) and a liquid-tight layer (5) on one side, and at least one adhesive element (6) for fastening the element (3) in a use position and for fastening the element (3) in its use position on the urine catheter (2), characterised in that the element (3) made of absorbent material (4) is in the form of a circle sector and, for its use position, can be rolled up to form a funnel and can be fixed in position using the at least one adhesive element (6), and in that at least one additional adhesive element (7) is arranged on a radius (r) of the circle sector for fastening to the urine catheter (2), which additional adhesive element (7) has an adhesive-free end (8).

2. The pad (1) according to claim 1, characterised in that the element in the form of a circle sector (3) has a central angle (a) of the circle sector of 90° to 270°, preferably 180°.

3. The pad (1) according to claim 1 or 2, characterised in that the radius (r₁) of the element in the form of a circle sector (3) is between 5 cm and 10 cm.

4. The pad (1) according to one of claims 1 to 3, characterised in that the area (A) of the element in the form of a circle sector (3) is between 20 cm² and 200 cm².

5. The pad (1) according to one of claims 1 to 4, characterised in that a cut-out (9) is provided in the region of the centre (M) of the element in the form of a circle sector (3).

6. The pad (1) according to claim 5, characterised in that the cut-out (9) is circular with a radius (r₂) of preferably 1 mm to 11 mm.

7. The pad (1) according to one of claims 1 to 4, characterised in that at least one incision (10) is provided in the region of the centre (M) of the element in the form of a circle sector (3).

8. The pad (1) according to claim 7, characterised in that the at least one incision (10) has a depth (t) of 1 mm to 11 mm.

9. The pad (1) according to one of claims 1 to 8, characterised in that the element in the form of a circle sector (3) is formed from highly absorbent plastic fibres, in particular acrylic or polyester fibres.

10. The pad (1) according to one of claims 1 to 9, characterised in that a gel-forming substance (11) is arranged in the element in the form of a circle sector (3).

11. The pad (1) according to one of claims 1 to 10, characterised in that the element in the form of a circle sector (3) has a contact mesh (12) on the side opposite the liquid-tight layer (5).

12. The pad (1) according to one of claims 1 to 11, characterised in that the adhesive element (6) for fastening the element in the form of a circle sector (3) in its use position is formed by a rounding.

13. The pad (1) according to one of claims 1 to 12, characterised in that the adhesive elements (6, 7) are formed in one piece.

14. The pad (1) according to one of claims 1 to 13, characterised in that the thickness (d) of the element in the form of a circle sector (3) is 1 mm to 9 mm, preferably 3 mm to 3.5 mm.
